(19) United States
(12) Patent Application Publication
(10) Pub. No.: US 2025/0170367 A1
(43) Pub. Date: May 29, 2025

(54) MULTI-LAYER OUTER COVER FOR BENDABLE MEDICAL DEVICES

(71) Applicant: CANON U.S.A., INC., MELVILLE, NY (US)

(72) Inventors: Zachary Hamilton Haubert, Boston, MA (US); Nathaniel Robert Lau, Boston, MA (US); Takahisa Kato, Brookline, MA (US); Benedict Shia, Needham, MA (US)

(21) Appl. No.: 18/841,990

(22) PCT Filed: Feb. 28, 2023
(86) PCT No.: PCT/US2023/014080

§ 371 Date: Aug. 27, 2024

Related U.S. Application Data
(60) Provisional application No. 63/314,903, filed on Feb. 28, 2022.

(51) Int. Cl. A61M 25/01; A61B 1/00; A61B 17/00; A61M 25/00; A61M 25/06

(52) U.S. Cl. CPC A61M 25/0144; A61M 25/0012; A61M 25/0045; A61M 25/0147; A61B 1/00135; A61B 2017/00336; A61M 2025/0681

(57) ABSTRACT

The present disclosure relates to a bendable medical device with a skeleton structure, driving wires, and a multi-layer outer cover. This multi-layer cover includes, from an interior face to an exterior face: an inner polymer tube which contacts the skeleton structure, a mesh tubular structure comprising a mesh which covers the inner polymer tube, and an outer polymer tube which contacts the mesh tubular structure.

### CROSS-REFERENCE TO RELATED APPLICATIONS

The present disclosure claims priority to U.S. provisional application 63/314,903 filed 28 Feb. 2022. The disclosures of the above-listed provisional application is hereby incorporated by reference in its entirety for all purposes. Priority benefit is claimed under 35 U.S.C. § 119 (e).

### FIELD OF THE DISCLOSURE

The present disclosure relates to medical devices. More particularly, the disclosure exemplifies embodiments of a covering the backbone structure of steerable medical devices, such as endoscopes or catheters.

### BACKGROUND OF THE DISCLOSURE

Bendable medical devices such as endoscopic surgical devices and catheters are well known and continue to gain acceptance in the medical field. The bendable medical device generally includes a flexible body commonly referred to as a sleeves or sheaths. One or more tool channels extend along (typically inside) the flexible body to allow access to a target located at a distal end of the body.

The bendable medical device is intended to provide flexible access within a patient, with at least one curve or more leading to the intended target, while retaining torsional and longitudinal rigidity so that a clinical user can control the tool located at the distal end of the medical device by maneuvering the proximal end of the device.

To be used in a clinical setting, the bendable medical devices must have an outer cover to protect the patient from the bendable medical device and any tools, effluents, etc. from the medical device. The cover also protects the bendable medical device from contamination and prevents buckling and reduces control failures.

There are currently multiple methods to attach an outer cover to a bendable medical device. Using heat to shrink down the polymers can deform or damage the delicate materials below in the bendable medical device application. Using a swelling agent is another alternative but is restricted to single layer materials that can swell. With this single layer, the mechanical properties are not sufficient to support the bendable medical device's wire penetration/buckling.

The bending section of bendable medical devices or endoscopess, for instance that described in U.S. Published Patent Application 2021/0369085, is composed of an inner liner, guide rings, drive wires which are anchored into guide rings at their distal end. The guide rings are attached on the inner liner with the intervals. This structure, named a skeleton structure, allows bending the medical device with tight curvature. The bendable medical device uniquely uses a pushing operation of the drive wires besides the conventional pulling operation.

The bending section also includes an outer cover to cover this skeleton structure. This tube mainly protects the internal structure from the external environment to avoid device malfunction, especially fluid ingress into the driving wires and the guide rings. The outer cover provides a smooth surface towards the anatomy of the patient to prevent compromised operation and potential harm to the anatomy.

However, this bending section has bending angle limitation issues due to wire buckling. The anchored drive wires experience both pushing and pulling during operation of the bendable medical device which causes bending of these backbone sections. The drive wire in the interval is basically unsupported without the guide structures and can have buckling with larger pushing force than its buckling limit. During bending, especially along the inner radius, the distance between the rings decreases and the opposite occurs along the outer radius. This increase in distance between the rings results in increased lengths of unsupported drive wires and reduces the buckling limit.

The wire buckling would cause bending control failure. It also potentially causes the outer cover to stretch and fail (e.g., puncture). Therefore, the pushing force to bend the skeleton structure is limited to be within this buckling limit, which would result in a limited bending angle with this configuration. Thus, there is needed a flexible medical device that has an outer cover that reduces or prevents wire buckling but yet can be fabricated and function with a bendable medical device to allow steering and function as a catheter or endoscope.

### SUMMARY

Thus, to address such exemplary needs in the industry, the presently disclosed apparatus teaches a bendable medical device. This device comprises a skeleton structure having a central lumen extrusion and a plurality of guide rings arranged on the outside of the central lumen extrusion, having spaced intervals between the guide rings; a plurality of driving wires configured pass through the guide rings, wherein the distal end of the plurality of driving wires are attached to the skeleton and the proximal end of the plurality of driving wires are configured to be mechanically connected to an actuator unit; and a multi-layer outer cover configured over the skeleton structure. The multi-layer outer cover comprises, from an interior face to an exterior face: an inner polymer tube which contacts the guide rings, a mesh tubular structure comprising a mesh which contacts and covers the inner polymer tube, and an outer polymer tube which contacts the mesh tubular structure and is configured to constrain the length of the mesh tubular structure.

Proximal to the skeleton structure, the bendable medical device may also have a more passive proximal tubular section that does not necessarily have guide rings. The driving wires pass through the passive proximal session, and the multi-layer outer cover is configured over the passive proximal tubular section. The mesh of the mesh tubular structure may be configured to increase the diameter of the mesh tubular structure as the length of the mesh tubular structure is decreased. Similarly, the mesh may be configured to also decrease the diameter of the mesh tubular structure as the length of the mesh tubular structure is increased.

In some embodiments, the mesh is a braided polyester tube.

In some embodiments, the bendable medical device includes an actuation unit that is connectable to the bendable medical device via the driving wires, where the actuation drives the driving wires in a push-pull configuration. In some embodiments, the skeleton structure has at least two bendable segments, or at least three bendable segments, each segment bendable by at least one driving wire. In some embodiments, each segment is bendable by three driving wires.

Also provided herewith is a method of manufacturing a multi-layer outer cover for a bendable medical device wherein the bendable medical device comprises a plurality of guide rings at an interval forming a skeleton structure, comprising the steps of:
a. placing an inner polymer tube over the plurality of guide rings and the intervals between the guide rings in the skeleton structure;
b. compressing a length of a mesh tubular structure and placing the mesh tubular structure over the central lumen extrusion, wherein compression of the length of the mesh tubular structure increases the diameter of the mesh tubular structure in comparison to the neutral diameter of the mesh tubular structure;
c. releasing the compressing force and aligning the mesh tubular structure to fit over central lumen extrusion; and
d. placing an outer polymer tube over the mesh tubular structure to constrain the position and the length of the mesh tubular structure.

In some embodiments, each of the inner polymer tube, the mesh tubular structure, and the outer polymer tube (the layered tubes) are in an interference fit with the layer below. Each layered tube may be expanded and contracted along its length within the constraints of the next outer layered tube. The methods as provided herein may also include a step of cauterizing the ends of the mesh tubular structure prior to placing the outer polymer tube.

### BRIEF DESCRIPTION OF THE DRAWINGS

The accompanying drawings, which are incorporated herein and form part of the specification, illustrate various embodiments, objects, features, and advantages of the present disclosure.

FIG. 1(A) illustrates an example embodiment of a medical system 1000 including a bendable medical device 11 in an applicable medical environment thereof. FIG. 1(B) illustrates an example embodiment of the medical system 1000 in bock diagram form;

FIG. 2(A) and FIG. 2(B) illustrate structural details of the bendable body of a bendable medical device having a central lumen and guide rings.

FIG. 3 is a photograph depicts a wire buckling in a bendable medical device.

FIG. 4 is a cross-sectional view of a steerable bendable medical device according to the present disclosure.

FIG. 5(A) is a cut-away view of the flexible medical device (not to scale) depicting the three-layer sandwich design of the multi-layer outer cover according to the present disclosure. FIG. 5(B) is a similar cut-away view of the flexible medical device (not to scale) depicting the three-layer sandwich design of the multi-layer outer cover that also has a three-layer sandwich design on the inner layer according to the present disclosure.

FIG. 6 is a diagram depicting the forces impacting the steerable bendable medical device's driving wires during bending.

FIG. 7 is a flow chart providing a manufacturing process.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative exemplary embodiments. It is intended that changes and modifications can be made to the described exemplary embodiments without departing from the true scope and spirit of the subject disclosure as defined by the appended claims.

### DETAILED DESCRIPTION OF EXEMPLARY EMBODIMENT

The present disclosure has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

The following paragraphs describe certain explanatory embodiments of a robotic medical system configured to use a bendable medical device, and particularly a steerable catheter. Other embodiments may include alternatives, equivalents, and modifications. Additionally, the explanatory embodiments may include several features, and a particular feature may not be essential to some embodiments of the devices, systems, and methods that are described herein.

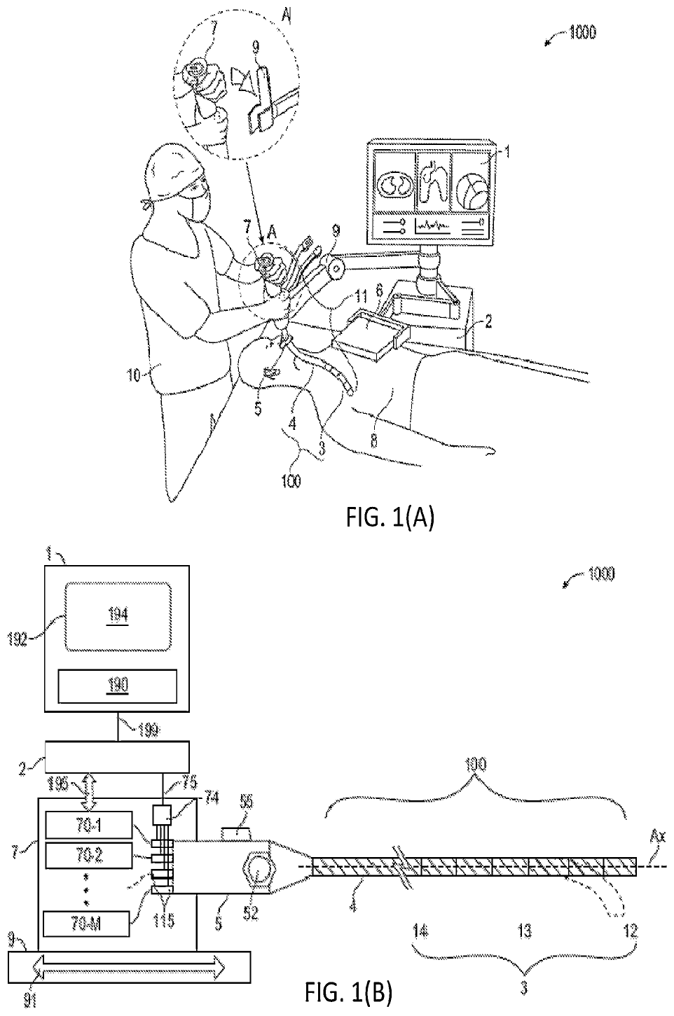

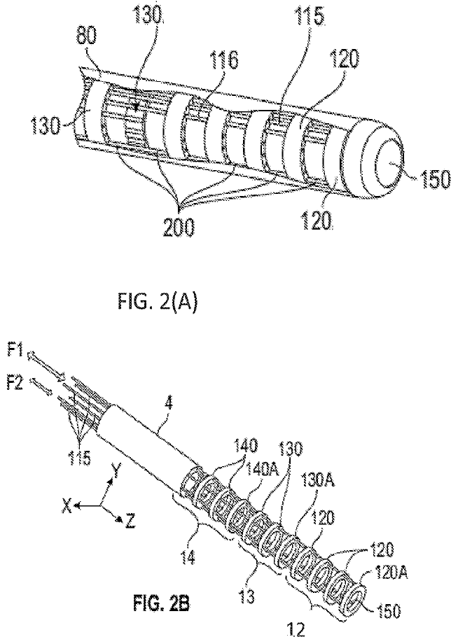

First, structural components of a robotic medical system 1000 comprising a bendable body 3 detachably attached to an actuation unit 7 via a connector assembly 5 will be described with reference to FIG. 1A, FIG. 1B, and FIG. 2A-2B. The robotic medical system 1000 can include a continuum or multi-segment robot configured to form a continuously curved geometry by actuating one or more bending sections of the bendable body 3. An example of a continuum robot is a snake-like endoscopic device, as described in applicant's previously published U.S. Pat. No. 9,144,370, and patent application publications US 2015/0088161, US 2018/0243900, US 2018/0311006 and US 2019/0015978, which are incorporated by reference herein for all purposes.

A robotic medical system will be described by referring to FIG. 1A and FIG. 1B. FIG. 1A illustrates an example embodiment of a medical system 1000 in a medical environment such as an operating room (OR). The medical system 1000 makes use of a bendable medical device 11 (steerable medical device) to treat a patient 8 under interactive commands of a user (e.g., a physician) 10. The medical system 1000 includes at least a navigation system 1, a controller system 2, and the bendable medical device 11. The bendable medical device 11 includes an actuation unit 7 and a steerable catheter sheath 100. The steerable catheter sheath 100 includes a multi-segment distal section 3 and a single-segment proximal section 4. The proximal section 4 is connected to the actuation unit 7 via a connector assembly 5. The actuation unit 7 is configured to be detachably mounted to a robotic platform (support platform) 9, as shown in detail on the inset A of FIG. 1A.

The bendable medical device 11 can be configured for a number of medical applications and/or industrial applications. Under medical applications, the bendable medical device 11 can be configured as a robotic endoscope, as a steerable catheter, as a surgical introducer sheath or sleeve that uses principles of kinematic (robotic) navigation for guiding a medical tool through tortuous bodily lumens. Robotic endoscopes can be used for a variety of different diagnostic and interventional procedures including, but not limited to, colonoscopy, bronchoscopy, laparoscopy, video endoscopy, etc. In the case of a video endoscope, the bendable medical device 11 would be configured with a miniature video camera, such as a CCD or CMOS camera, positioned at the distal portion of the bendable body 3, as well as electronic cabling and illumination optics (an optical fiber) extending along the tool channel.

FIG. 1B illustrates an example embodiment of the medical system 1000 in functional block diagram. The catheter sheath 100 has a proximal non-steerable section 4, and a distal steerable section 3 made of the multiple bending segments (e.g., bending segments 14, 13, 12) which are arranged lengthwise along a longitudinal axis (Ax). At least one central lumen or tool channel extends along the length of the catheter sheath 100 and through part of the connector assembly 5. In at least some embodiments, the bendable medical device 11 is controlled by a robotic controller system 2 via the actuation unit 7; the actuation unit 7 is a handheld controller (handle) connected to the proximal section 4 of the catheter sheath 100 by or connector assembly 5. The actuation unit 7 can include any force generating device and a mechanical element respectively used to generate and transmit sufficient actuating force for bending at least one bending segment of the steerable section 3. In that regard, actuation unit 7 may include any device capable of generating and transmitting an actuating force including, for example, a mechanical force, hydraulic force, magnetic force, or pneumatic force. The support platform 9 may include, for example, a robotic arm and a linear stage 91 which serves to guide the bendable medical device 11 (control unit 7, connector assembly 5 and catheter sheath 100) in a moving direction (typically linear movement) for insertion and/or retraction of the catheter sheath 100 with respect to the patient 8.

The controller system 2 generally includes electronic components such as a PID controller and/or a digital signal processor (DSP) device along with suitable software, firmware and peripheral hardware, which are generally known per se to persons having ordinary skill in the art. The controller system 2 can be part of, or is connected to, the navigation system 1 (e.g., a computer or system console). The navigation system 1 includes the necessary software (computer-executable code, programs and applications) executable by a central processing unit (CPU) 190, according to a user's interactions with the system 1000 via a user interface 194, to control the bendable medical device 11. Operation of CPU 190 may be implemented by one or more processors in a computer loading and executing a program, or may be implemented by a dedicated circuit (FPGA and ASIC). The user interface 194 may include, for example, a display device 192 (LCD, LED or OLED display) which may include a graphical user interface (GUI) and/or a pointing device and keyboard (not shown), or a touchscreen.

The navigation system 1, the controller system 2, and the actuation unit 7, are operably connected to each other by a network connection or a cable bundle 199 and a data bus system 195. Among other functions, the navigation system 1 can provide a surgeon or other user with a GUI and other information displayed in the image display device 192, so that the user can interact and remotely operate the bendable medical device 11.

The controller system 2 is configured to control the actuation unit 7 which includes a plurality of actuating motors (or actuators) 70-1, 70-2 . . . , 70-M. The number of actuators or motors 70 will depend on the design of the actuation unit 7, and it can include a single (one) actuator or motor that can actuate all driving wires independently, or it could include a number of actuators or motors equal to a number of driving wires 115 so that each actuator or motor can actuate each driving wire individually.

The controller system 2 may also include or be connected to one or more sensors 74. Sensors 74 can include a strain sensor and/or a position sensor which are configured to detect and/or measure compressive or tensile forces (actuating forces) exerted on the driving wires 115 to bend one or more of the segments 12, 13 and 14. Sensors 74 may output a signal 75 corresponding to an amount of compressive or tensile force (an amount of strain) being applied to a driving wire 115 at any given point in time. The signals 75 from the sensors 74 (strain sensor and/or position sensor) for each driving wire are fed into the controller system 2 to control each actuator individually. In this manner, each driving wire can be actively controlled, by a feedback loop, to implement appropriate shaft guidance for navigating the steerable section 3 through intraluminal tortuous paths of a patient's anatomy.

FIG. 2A and FIG. 2B illustrate additional details of the catheter sheath 100, according to an embodiment of the present disclosure. Most of these robotically steerable medical devices have polymer rings 120 arranged around a central lumen 150 so as to create a flexible backbone for snake-like articulation. Therefore, this type of steerable medical instruments is known as a snake or continuum robot. The snake-like continuum robot has a unique distal structure wherein the polymer rings are attached to the central lumen at a predetermined spacing to form a skeleton structure with specific bending properties. The central lumen 150 can be a single lumen extrusion made of a low durometer material to reduce the forces required to bend the skeleton structure. The single lumen skeleton structure made of low durometer material can achieve a relatively tight bending radius. As the catheter sheath is bent into a curved shape, the gap between the rings increases on the outer radius and decreases on the inner radius of the curved structure.

FIG. 2A is a 3D rendering and FIG. 2B is a perspective view of the catheter sheath 100 comprised of a non-steerable proximal section 4 and a steerable distal section 3. The steerable section 3 incudes a plurality of bending segments comprising a proximal bending segment 14, a middle bending segment 13, and a distal bending segment 12. As shown in FIG. 2B, each bending segment is formed of two or more rings (a plurality of rings) cooperatively arranged in a lengthwise direction to form a tubular structure. As shown in FIG. 2A, the tubular structure also includes an outer cover 80 which is partially shown and a central lumen extrusion 200. The outer cover is a multi-layer outer cover having a tubular shape, and will be described in more detail below.

The inner liner, or central lumen extrusion 200 has an inner surface which defines a central lumen or tool channel 150, and an outer surface onto which a plurality of rings are arranged. The rings include a plurality of wire conduits (secondary lumens) through which driving wires 115 and/or support wires 116 are passed. The driving wires 115 are moved by an actuating force to bend one or more segments of the steerable section; the support wires 116 are not actuated.

At least some rings have secondary lumens used as conduits for control wire or support wires to actuate the distal end of the catheter sheath. The outer surface of the central lumen extrusion and/or the inner surface of the rings are specifically designed to achieve tight bending of the catheter sheath in tortuous anatomies having curvatures greater than 90 degrees. The spacing of the rings, also provides for the high curvature of the catheter sheath.

FIG. 2B illustrates an example of the catheter sheath 100 where the central lumen extrusion 200 and the outer cover 80 are not displayed in the image. As shown in FIG. 2B, the plurality of driving wires 115 pass through the proximal section 4, advance through wire conduits of wire-guiding rings 140 of the proximal bending segment 14, pass through wire conduits of wire-guiding rings 130 of the middle bending segment 13, and pass through wire conduits of wire-guiding ring 120 of the distal bending segment 12. (as shown in this figure, the guide rings may be defined all similarly as guide rings 120, or may be defined based on which of the multiple separate bending sections 12, 13, 14 as guide rings 120, 130, and 140.) Each bending segment of the steerable section is actuated by a set of antagonistic driving wires 115 which operate by a pulling or pushing force (an actuating force) to bend each bending segment independently from each other. Forces F1 and F2 of different magnitude can be applied in the lengthwise direction to separate driving wires to bend the various bending segments in desired directions. A combination of forces F1 and F2 can also be applied to bend a given bending segment in additional directions. To that end, a first set of driving wires 115 may be anchored at an anchor ring 120A at the distal end of the distal segment 12, a second set of driving wires 115 may be anchored at the anchor ring 130A of the middle bending segment 13, and at a third set of driving wires 115 may be anchored at the anchor ring 140A of the proximal bending segment 14.

According to one embodiment, three driving wires 115 may be used to actuate each bending section. In that case, the distal ends of the driving wires 115 in the first set of driving wires can be anchored to anchor ring 120A, the second set of driving wires can be anchored to the anchor ring 130A, and the third set of driving wires can be anchored to the anchor ring 140A. In such example, nine driving wires 115 will pass through the proximal section 4 of the steerable sheath. At each anchor member, it may be advantageous to arrange (to anchor) the driving wires 115 equidistantly around the circumference of each anchor member at strategic locations so as to actuate each bending segment independently in a desired direction. For example, each driving wire 115 can be anchored at equal intervals on the anchor member, e.g., when each bending segment is actuated by three wires, the driving wires would be anchored at 120-degree intervals to be able to actuate each bending segment in substantially any direction (any angle with respect to lumen axis Ax).

As shown in FIG. 2A and FIG. 2B, in the catheter sheath, each bending segment 12, 13, and 14 includes a plurality of ring-shaped wire-guiding members (guide rings), while the proximal non-steerable section 4 is a single piece elongated tubular component. Here, the tubular shaped passive proximal section 4 and the central lumen extrusion 200 can be made of similar biocompatible polymer materials. Additionally, the outer cover 80 can be made of biocompatible polymer materials. These materials include, but are not limited to polyether block amide copolymer (e.g., Pebax® brand produced by Arkema), which is a well known polymer used in the fabrication of catheter shafts. Other medical-grade thermoplastic polyurethane (TPU) and thermoplastic elastomer (TPE) materials can also be used as tubing extrusion materials for medical catheter and endoscope devices that demand precision and consistency. Furthermore, other commonly known catheter tubing materials may be used, including PVC, HDPE, Polyurethane, Nylon, FEP, PFA, ETFE, PTFE (liners), PEEK, TPE, silicones, Grilamid® lubricious films, and many others. The biocompatible polymer materials used in the central lumen extrusion 200, the proximal non-steerable section 4, and the outer cover 80 may be the same or similar polymer but selected for the different needs in hardness, lubricity, and swellability needed for the specific use.

Referring back to FIG. 1A and FIG. 1B, the handle or connector assembly 5 provides an electromechanical interface between the proximal section 4 and the actuators in actuation unit 7. For example, the connector assembly 5 may provide mechanical, electrical, and/or optical connections, and other data/digital connections for interfacing the bendable medical device 11 with the controller system 2 and the navigation system 1. The handle or connector assembly 5 may also provide an access port 55 which can be used by a surgeon or other operator to insert instruments or end effectors through the tool channel 150. For example, the access port 55 can be used to insert small instruments, such as small forceps, needles, or electrocautery instruments and the like. In addition, the connector assembly 5 may include one or more dials or control wheels 52 for manual control (bending or steering) of at least one segment of the steerable section. In some embodiments, the bendable body 3 may include more that one tool channel 150, where at least one of those channels can be used for passing liquid and/or gaseous fluids, and another channel can be used for passing tools or imaging devices.

In operation, the navigation system 1 and the controller system 2 are communicatively-coupled via the data bus 199 to transmit and receive data to and from each other. The navigation system 1 is also connected to, and communicates with, external equipment such as a computed tomography (CT) scanner, a fluoroscope imager, an image server (not shown in FIG. 1A), etc., which are external of the medical system 1000. The image server may include, but is not limited to, a DICOM™ server connected to a PACS (Picture Archiving and Communication System) or medical imaging system which may include, but is not limited to, one or more of the CT scanner, a magnetic resonance imaging (MRI) scanner, or a fluoroscope, etc. The navigation system 1 processes data provided by the controller system 2, data provided by images stored on the image server, or data provided by images from the CT scanner or the fluoroscope. The navigation system 1 displays images and other medical information in an image display device 192 to aid the user 10 in performing a medical procedure.

For a medical procedure where the bendable medical device 11 will be used, medical images (e.g., from the CT scanner) are pre-operatively provided to the navigation system 1. With the navigation system 1, a clinical user creates an anatomical computer model from the images. In a particular example embodiment of FIG. 1A, the anatomy can be the lung airways of patient 8. From chest images received from the CT scanner or PACS system, the clinical user can segment the lung airways for clinical treatments, such as a biopsy. After the navigation system 1 generates a map of the lung airways, the user can also use the navigation software system to create a plan to access a lesion for the biopsy. The plan includes the target lesion and a trajectory (navigation path) through the airways to insert the bendable body 3 (steerable sheath) of the bendable medical device 11.

The controller system 2 includes firmware, control circuitry and peripheral hardware to control the bendable medical device 11, the insertion unit 9, and a field generator 6 (e.g., an electromagnetic (EM) field generator). The controller system 2 is communicatively coupled with the actuation unit 7, the insertion unit 9, the EM field generator 6, and a man-machine interface (e.g., a gamepad controller not shown in FIG. 1A-FIG. 1B). In this manner, the controller system 2, in coordination with the navigation system 1, controls the overall functions of the bendable medical device 11 and the insertion unit 9.

The bendable medical device 11 includes the bendable body 3, the handle or connector assembly 5, and the actuation unit 7. The actuation unit 7 is configured to bend one or more the proximal bending segment 14, the middle bending segment 13, and the distal segment 12 via the connector assembly 5 according to commands from the controller system 2, and based on the navigation plan provided by navigation system 1.

According to one embodiment, either during insertion or retraction of the bendable body 11, the controller system 2 may control the linear stage 91 of insertion unit 9 to move the bendable body 3 along the center line of a lumen (e.g., an airway) in a desired trajectory followed by active control of the bending segments. This is similar to known shaft guidance techniques used to control robotic guided catheters or endoscopes with the goal of forcing the flexible shaft of the sheath to keep to a desired trajectory. In one example, when using the navigation system 1, the bendable medical device 11 is robotically controlled to advance the sheath through a lumen while sensors 74 measure the actuation force, insertion depth, the angulations of user-controlled steerable segments, etc., to obtain trajectory information. The trajectory information is stored in a memory of the system and continuously updated. After a short advance in insertion or retraction distance, the shape of the bendable body 3 is corrected by adjusting (actuating) one or more of the bending segments in such a way that the new shape closely matches the desired trajectory. This process is repeated until a target area is reached. The same process can be applied when the bendable body is controlled to withdraw the bendable body 3 from the patient. This process is similar to the navigation process described in, e.g., US 2007/0135803, which is incorporated by reference herein for all purposes. Additional details for driving a snake-like robot include the control methods for actuation, as described in applicant's previous patent application publications US 2015/0088161, US 2018/0243900, US 2018/0311006, and US 2019/0015978, which are also incorporated by reference herein for all purposes. To improve the navigation process, it is advantageous to reinforce the inner liner of the central lumen or tool channel 150.

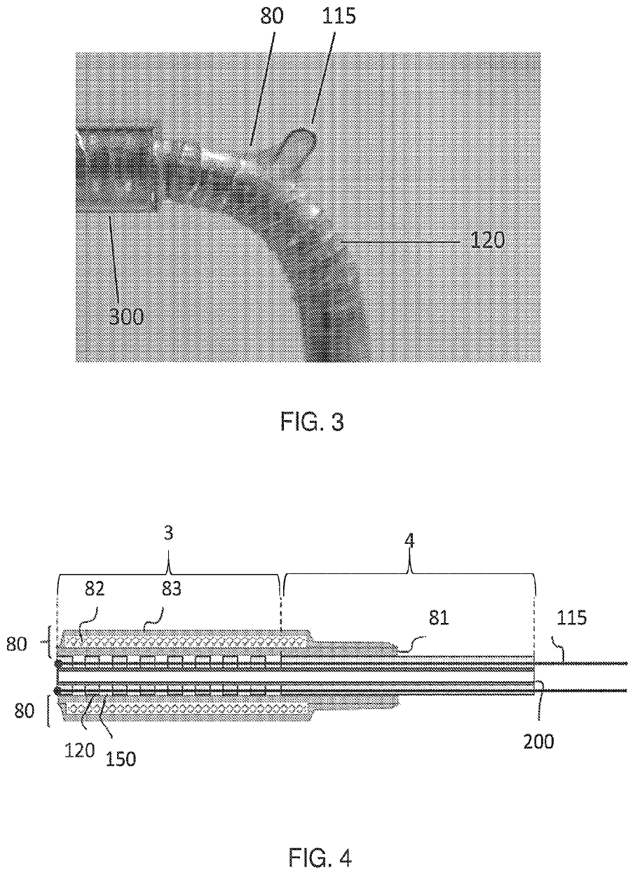

FIG. 3 provides an illustration where a portion of the flexible medical device is partially outside of an external lumen 300. In this illustrative embodiment of the problems in the art, the driving wire 115 has buckled and can no longer translate bending for to the guide rings 120. Thus there is a loss of control of the device and potential for puncture in the wall which can damage the catheter or lead to patient harm. The single polymer layer outer cover 80 on the flexible medical device of this embodiment can be seen as still covering the buckled driving wire 115. However, this single-layer outer cover 80 is not be sufficient to prevent buckling and the problems associated with buckling.

A cross-sectional view of the flexible medical device of this disclosure is shown in FIG. 4. The guide rings 120 are attached on the central lumen extrusion 200 with the intervals 150. The driving wires 115 are terminated at the distal end of the steerable medical device. The guide rings 120 and the central lumen extrusion 200 form the skeleton structure with spaces 150 between the various guide rings 120. With the driving wires 115, this skeleton structure creates the bending section 3.

In some embodiments, proximal to the bending section, there is a passive proximal tubular section 4. This section has a central lumen or tool channel 150 central lumen extrusion 200. The driving wires 115 also run through this passive proximal section and are connected to the actuation methods to push or pull to bend the bending section. For use of the flexible medical device in the lung, the passive proximal tubular section is long enough to access the bronchi through the trachea and is relatively rigid compared to the skeleton structure. The length and rigidity of this section can be adjusted based on the needed flexible medical device. The driving wires extend proximal to the proximal tubular section and are configured to attach to an actuator that will drive the driving wires in a push-pull configuration.

The outer cover 80 is a multi-layer outer cover that can be seen with a unique braided assembly, forming a three layer “sandwich outer cover” having an inner polymer tube 81, a mesh tubular structure 82 and an outer polymer tube 83. The inner polymer tube 81 contacts the guide rings, the mesh tubular structure 82 can be concentric with the inner polymer tube and comprise a mesh which contacts and covers the inner polymer tube. The outer polymer tube 83 contacts the mesh tubular structure 82 and may be configured to constrain the length of the mesh tubular structure. This sandwich outer cover covers all intervals between the guide rings in the bending section and can increase the buckling limit of the driving wires and prevent the wire buckling failures.

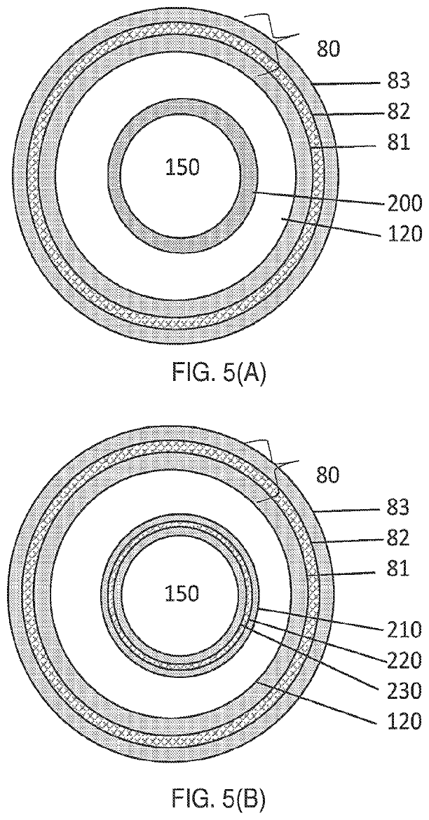

The outer cover 80 can also be seen in the cross-sectional view of the flexible medical device in FIG. 5(A). While this figure is not to scale, the various components are easy to see, starting with the central lumen 150 or tool channel surrounded by an inner liner 200 and the guide ring 120 (excluding any secondary lumen and driving wires at this location of the cross-section). Outside of the guide ring 120 is the outer cover 80 which is a sandwich structure, the outer cover having an inner tube 81, a mesh tubular structure 82, and an outer tube 83, each of which are arranged substantially concentric to the guide ring 120.

The inner tube 81 can be shrunk directly over the guide rings 120 with an interference fit. This tube will provide continuous high-friction surface to the next mesh tubular structure. The tube is flexible and can contract and extend. In this particular embodiment, the inner polymer tube is made of 80 A Pellethane. A lubricious additive may be compounded into the resin to provide a more lubricious outer surface for the inner tube 81.

Once in place, the inner tube 81 should have a high friction with the guide rings 120. This creates a high axial stiffness and helps maintain the guide ring interval gap. If there is insufficient friction between these two components or an inner tube was not used, the friction between the mesh tubular structure 82 and the guide rings 120 would not be strong enough and cause low axial stiffness, thus resulting in the guide ring interval gap to expand allowing for lower buckling forces in the medical device.

The mesh tubular structure 82 can be a braided tube, and can be composed of long fibers braided in a crisscross pattern. The pick count (pics per inch, or PPI) is represents the number of times the braid wire crosses for every inch of the shaft length. The PPI selected would provide sufficient radial resistance (hoop strength) to puncture when contained within the sandwich and still sufficient flexibility for catheter operation In some embodiments, the mesh tubular structure is a braided polyester tube. In some embodiments, it is an input yarn with a standard 1 over 2 configuration. In some embodiments the braided tube 82 is made out of the same material as the inner tube 82 (e.g., a polyurethane fiber with a higher durometer than the inner tube) that can expand and recover with the inner tube.

Diameter of the fibers of the braid is also selected based on flexibility and not increasing the diameter of the bendable medical device beyond the ability to operate in applicable procedures. Since this structure is applied separately, the inner/outer polymer tubes can be expanded or shrunk independently of the braided tube. Also, when the braid is applied independently it can be expanded or shrunk by either compressing or expanding the length of braid (“finger-trap” effect, where the braid diameter and tension are inversely proportional). This is because as the braid is lengthened, the angle of the wrap and weft threads decreases at their crossing points. This reduces the radial distance between opposing sides and thus the overall circumference of the braid. The opposite occurs when the braid is compressed, and this unique property of the braid solves the assembly issue of putting the braid into position over the backbone structure. In this particular embodiment, the braided tube is made of Polyester (PET).

The outer tube 83 is at the interface between the flexible medical device and the surrounding environment. In manufacture, the outer tube 83 can be a polymeric tube that is placed over both tubes in the same manner as the inner polymer tube layer 91. This outer tube 83 can hold the braided tube 82 in place and constrain the braided tube length by sandwiching it with the inner polymer tube 81. This outer tube 83 is also not permeable to blood or fluid, preventing liquid ingress into the bendable medical device. In this particular embodiment, the outer polymer tube 83 is made of the same 80 A Pellethane as the inner polymer tube 81. A lubricious additive may be compounded into the resin to provide a more lubricious outer surface for the outer polymer tube 83.

FIG. 5(B) shows a cross section of another embodiment of the flexible medical device as described herein. In addition to all the components as described in FIG. 5(A), the central lumen extrusion (200 in FIG. 5(A)) is composed of three parts as well, an inner surface 230, a reinforcing structure 220 and an outer surface 210. This structure for the central lumen extrusion is described in detail in U.S. Pat. Pub. 2022/0126060, herein incorporated by reference in its entirety.

### Function

The multi-layer (sandwich) outer cover provides rigid hoop strength and flexible bending at the same time. This unique combination of properties increases the buckling limit and allows for bending of the bending section at tight curvature with pushing driving wires.

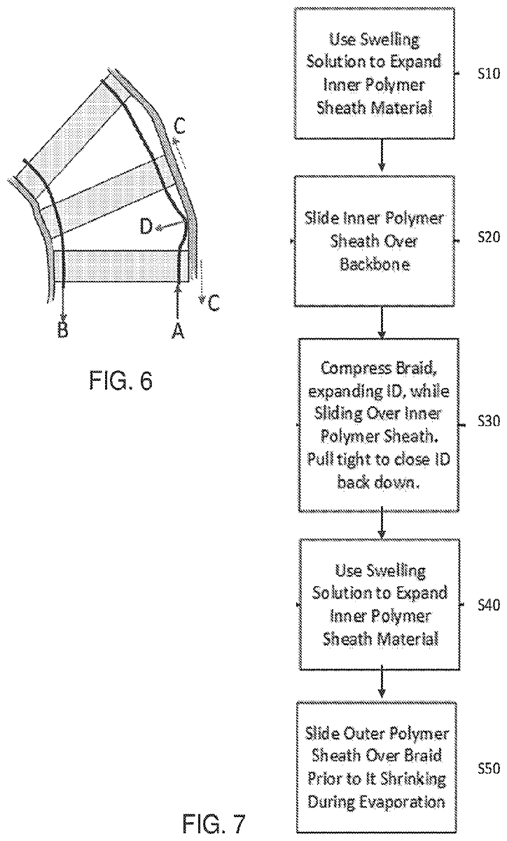

During the bending, the driving wire with the pushing force (arrow A in FIG. 6) is always in the outer arc side in the bending section. On the outer arc side, the driving wires would be subjected to a risk of catastrophic buckling since the intervals between the guide rings on the outer arc increases as the pushing forces increases.

In this disclosure, the braided tube in the middle of the sandwich outer cover has an important role for the above-mentioned benefit by providing the “finger-trap” effect. While the braided tube has a rigid hoop strength when the length is fixed, the diameter of the braided tube will decrease as the length is increased by the tensile forces. During bending, the braided tube on the outer arc side is subjected to the tensile force (arrows C) and is pushing back the driving wire from starting to herniate under the pushing force (arrow D) by reducing the diameter with the “finger-trap” effect (FIG. 6). This effectively prevents catastrophic buckling and allows the continuation of pushing the driving wire to bend the bending section. Additionally, the inner and outer polymer tubes have unique functions during this process. The inner polymer tube may be an expandable film with high-friction surfaces on both sides. Embodiments are the inner polymer tube are described in U.S. Pat. Pub. 2022/0126,060, herein incorporated by reference. The inner polymer tube transfers tensile forces (arrow C) by the adjacent guide rings to the braided tube. With the high-friction surface, the inner polymer tube can grip the guide rings and the braid tubes. The outer polymer tube is also an expandable film with high-friction surface on an inner side and covers the full length of the braided tube creating the sandwich structure (see FIG. 5). This structure holds the braided tube in place and makes the “finger-trap” effect occur in reaction to bending, to allow using the tensile force (arrow C of FIG. 6) to use the elongation of the braided tube. The outer surface of the outer polymer tube can be a smooth surface adjacent to the anatomy to promote smooth insertion of the bendable medical device.

One alternative design is to replace either the outer or inner polymer layer with an applied adhesive or coating layer creating the sandwich design.

There are multiple advantages of the presently claimed disclosure. One of these advantages is that the inner layer of polymer sheath holds the ring portion of the skeleton structure in place and creates column stiffness. This allows the ring gaps to be minimized under the wire translational pushing force, which in turn, allows for a maximized bend radius.

Another advantage of the presently claimed disclosure is that as the inner sheath connects the braid and the rings, it allows for a tight friction fit on skeletal section with the support of the braid, which has a spring like effect. The outer layer of polymer sheath can hold the braid tightly against the bendable medical device. This keeps everything assembled and holds the braid in tension. Also, this maximizes the braid angle for hoop strength by resist diameter increases.

Since the polymer sheath must be a medical grade polymer for medical applications and since it is important to minimize the overall diameter of the bendable medical device, the addition of the braid, which can have low stretch and much stronger anti-puncture characteristics than the polymer sheath, is advantageous. Also, minimally increases to bending stiffness are added when including sandwich structure including the braid, particularly when compared to axial support when compared to other methods such as only braided tubes.

The combination of the braid sandwiched between the polymer sheath layers convert radial forces into axial force along the bendable medical device. As axial force is delivered to the outer cover the braid angle opens which causes constriction (axial force) along the length of the bendable medical device. Further, during bending, as braid elongates around outer edge, the braid increases the inward axial force which resists wire force and resists penetration of the wire through the outer cover. Wire forces would be seen around the outer edge of a bend aligning with this phenomenon. Since the bendable device may be used within, for example, a lung or kidney, maintaining the structural integrity of the device to prevent wire penetration is important for the safety of the surrounding organ. It also keeps fluids out of the structure of the bendable medical device.

Another advantage of the present invention is that the sandwich structure helps to distribute bending forces of the skeleton structure along multiple rings and provides a better bending arc.

In referring to the description, specific details are set forth in order to provide a thorough understanding of the examples disclosed. In other instances, well-known methods, procedures, components and circuits have not been described in detail as not to unnecessarily lengthen the present disclosure.

### Manufacturing

In general, placing a tube having rigid hoop strength over the skeleton structure requires being able to either expand the tube and pulling it over the entire assembly before releasing or, requires using an oversized tube and shrinking the tube onto assembly once it is in position. The tube with rigid hoop strength, like the mesh tubular structure described herein, can be difficult to be expanded to radial direction and cannot be shrunken or expanded easily without causing permanent damage to the braided structure. This is because mesh tubular structures are produced as a single unit so significant OD changes would cause braid to damage the polymer material. Also heating dissimilar materials in a heat shrink type application would be difficult with the dissimilar materials.

The “finger-trap” effect of the mesh tubular structure in this invention, which involves diameter changes when you expand and contract length of the mesh or braid, can solve this manufacturing difficulty of assembling a long sheath structure over an elongated bendable medical device. The mesh tubular structure with the “finger-trap” effect can increase the diameter by compressing the length when the mesh tubular structure is applied on the assembly, then the diameter can be reduced to fit the assembly by releasing the compression force, if necessary, by extending the length. Due to assembly issues, braided outer covers cannot give the same outcome as the sandwich and often, braided catheters cannot be fabricated with heat or expanded.

The sandwich structure with the inner and outer tubes can allow the length of the mesh tubular structure to be maintained with the best fitting diameter over the skeleton structure. In such an application, the length of the mesh tubular structure can be constrained by the inner and outer layers being joined together either holding the mesh tubular structure in place, or the inner and outer layers can be joined beyond the edge of the mesh tubular structure, providing a defined space within which the mesh tubular structure can lengthen and shorten in response to bending of the bendable medical device. FIG. 7 provides an example manufacturing flow of the sandwich outer cover.

In the embodiment shown in FIG. 7, the inner polymer tube (or sheath) is first placed and creates a tight interference fit, tying the guide rings to the inner polymer tube. This supports the skeleton structure and distributes the bending forces. This layer serves as the direct interface to the driving wires when they are exhibiting axial forces. Also, like the outer polymer tube, this layer prevents fluid ingress into the bendable medical device. To place the inner polymer tube, a swelling solution is used to expand the inner polymer sheath material S10. The inner polymer tube is then slid over the backbone, S20. As the swelling agent dries, the inner polymer tube shrinks in diameter, creating an interference fit against the rings.

Then, the mesh tubular structure is compressed in length in order to expand the inner diameter while it is put over the inner polymer tube. The compression is released such that the mesh tubular structure is then pulled tight in length to close the inner diameter onto the layer below S30. At minimum the compression is released to the free state. However, for optimal results, tension may then be applied to the mesh tubular structure such that the diameter of the mesh tubular structure increases, creating an interference fit with the catheter and further increasing the hoop strength. Then, when the outer polymer tube is then applied on top of the mesh tubular structure, the outer polymer tube is holds the braided layer in this tension state.

In some embodiments, the braid is a 100 denier input yarn with a standard 1 over 2 configuration. This yarn, with a diameter of 60.0 +_−0.5 is 3.5 in free state and expands to a minimum of 3.9 under compression.

After that, the outer polymer tube (sheath) is applied over the mesh tubular structure by using swelling solution to expand the inner polymer sheath material S40. Finally, the outer polymer sheath is slid over the braid prior to it shrinking during evaporation S50. The application of the outer polymer tube is done with a solvent that causes the outer polymer tube to swell and to expand as it absorbs the solvent. As the solvent flashes off, the outer polymer tube returns to its original dimensions. The solvent used in an embodiment having a polyurethane outer polymer tube may be specific to polyurethanes; similarly, if a silicon outer polymer tube is used, a solvent for swelling silicones could be used.

The swelling used to apply either the inner polymer tube and outer polymer tube are not necessarily controlled during the process, so the tube swells larger than the minimum to be able to be loaded over the skeleton. However, in some embodiments, the swelling is limited to the amount needed to fit over the skeleton. In these embodiments, a metal braid could be able to adjust its braid angle along with the expansion of the base tube, and then recover along with it.

Terminating the edges of the mesh tubular structure could be performed, for example, by simply leaving the polymer sheath layers longer than the braid layer and sealing these two layers together. The braid ends themselves could be, for example, left free, welded together, cauterized or potted.

While this is one example for the manufacture of the several embodiments as described herein, it is understood that there can be many variations and different methods for manufacture, and the present embodiments are not limited to the methods disclosed.

### Definitions

Throughout the figures, where possible, the same reference numerals and characters, unless otherwise stated, are used to denote like features, elements, components or portions of the illustrated embodiments. In addition, while the subject disclosure is described in detail with reference to the enclosed figures, it is done so in connection with illustrative exemplary embodiments. It is intended that changes and modifications can be made to the described exemplary embodiments without departing from the true scope of the subject disclosure as defined by the appended claims. Although the drawings represent some possible configurations and approaches, the drawings are not necessarily to scale and certain features may be exaggerated, removed, or partially sectioned to better illustrate and explain certain aspects of the present disclosure. The descriptions set forth herein are not intended to be exhaustive or otherwise limit or restrict the claims to the precise forms and configurations shown in the drawings and disclosed in the following detailed description.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly. Similarly, the relative spatial terms “proximal” and “distal” may also be interchangeable, where applicable.

The term “about,” as used herein means, for example, within 10%, within 5%, or less. In some embodiments, the term “about” may mean within measurement error.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “includes”, “including,” and “containing” are to be construed as open-ended terms (i.e., meaning “including, but not limited to,”) unless otherwise noted. Specifically, these terms, when used in the present specification, specify the presence of stated features, integers, steps, operations, elements, and/or components, but do not preclude the presence or addition of one or more other features, integers, steps, operations, elements, components, and/or groups thereof not explicitly stated. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

The present disclosure generally relates to medical devices, and it exemplifies embodiments of a steerable catheter sheath for guiding a catheter and/or an optical probe which may be applicable to an imaging apparatus (e.g., an endoscope). The imaging apparatus may image using a miniature camera based on chip-on-tip (COT) technology, or may provide some other form of imaging such as spectrally encoded endoscopy (SEE) imaging technology (see, e.g., U.S. Pat. Nos. 10,288,868 and 10,261,223). In some embodiments, the imaging apparatus may include an optical coherence tomographic (OCT) apparatus, a spectroscopy apparatus, or a combination of such apparatuses (e.g., a multi-modality imaging probe).

The embodiments of the bendable medical device and portions thereof are described in terms of their positon/orientation in a three-dimensional space. As used herein, the term “position” refers to the location of an object or a portion of an object in the three-dimensional space (e.g., three degrees of translational freedom along Cartesian X, Y, Z coordinates); the term “orientation” refers to the rotational placement of an object or a portion of an object (three degrees of rotational freedom--e.g., roll, pitch, and yaw); the term “posture” refers to the position of an object or a portion of an object in at least one degree of translational freedom and to the orientation of that object or portion of object in at least one degree of rotational freedom (up to a total six degrees of freedom); the term “shape” refers to a set of posture, positions, and/or orientations measured along the elongated body of the object. As it is known in the field of medical devices, the terms “proximal” and “distal” are used with reference to the manipulation of an end of an instrument extending from the user to a surgical or diagnostic site. In this regard, the term “proximal” refers to the portion of the instrument closer to the user, and the term “distal” refers to the portion of the instrument further away from the user and closer to a surgical or diagnostic site.

As used herein the term “catheter” generally refers to a flexible and thin tubular instrument made of medical grade material designed to be inserted through a narrow opening into a bodily lumen (e.g., a vessel) to perform a broad range of medical functions. The catheter may be solely an imaging apparatus or it may comprise tools for use in therapeutic or diagnostic procedures. The more specific term “optical catheter” refers to a medical instrument comprising an elongated bundle of one or more flexible light conducting fibers disposed inside a protective sheath made of medical grade material and having an optical imaging function. A particular example of an optical catheter is fiber optic catheter which comprises a sheath, a coil, a protector and an optical probe. In some applications a catheter may include a “guide catheter” which functions similarly to a sheath.

As used herein the term “endoscope” refers to a rigid or flexible medical instrument which uses light guided by an optical probe to look inside a body cavity or organ. A medical procedure, in which an endoscope is inserted through a natural opening, is called an endoscopy. Specialized endoscopes are generally named for how or where the endoscope is intended to be used, such as the bronchoscope (mouth), sigmoidoscope (rectum), cystoscope (bladder), nephroscope (kidney), bronchoscope (bronchi), laryngoscope (larynx), otoscope (ear), arthroscope (joint), laparoscope (abdomen), and gastrointestinal endoscopes.

It will be appreciated that the methods and compositions of the instant disclosure can be incorporated in the form of a variety of embodiments, only a few of which are disclosed herein. Variations of those embodiments may become apparent to those of ordinary skill in the art upon reading the foregoing description. Skilled artisans are expected and understood to employ such variations as appropriate, and the present disclosure is intended to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A bendable medical device comprising:
a skeleton structure having a central lumen extrusion and a plurality of guide rings arranged on the outside of the central lumen extrusion, having spaced intervals between the guide rings;
a plurality of driving wires configured pass through the guide rings, wherein the distal end of the plurality of driving wires are attached to the skeleton and the proximal end of the plurality of driving wires are configured to be mechanically connected to an actuator unit; and
an outer cover configured over the skeleton structure, wherein the outer cover comprises, from an interior face to an exterior face:
an inner polymer portion which contacts the guide rings,
a mesh tubular structure, and
an outer polymer portion which is configured to constrain the length of the mesh tubular structure and has a smooth outer surface.

2. The bendable medical device of claim 1, further comprising a passive proximal tubular section which is proximal to the skeleton structure, wherein the driving wires pass through the passive proximal session, and wherein the multi-layer outer cover is configured over the passive proximal tubular section.

3. The bendable medical device of claim 1, wherein the mesh of the mesh tubular structure is configured to increase the diameter of the mesh tubular structure as the length of the mesh tubular structure is decreased.

4. The bendable medical device of claim 3, wherein the mesh of the mesh tubular structure of the outer cover is configured to also decrease the diameter of the mesh tubular structure as the length of the mesh tubular structure is increased.

5. The bendable medical device of claim 1, wherein the mesh is a braided polyester tube.

6. The bendable medical device of claim 1, further comprising an actuation unit that is connectable to the bendable medical device via the driving wires, where the actuation unit drives the driving wires in a push-pull configuration.

7. The bendable medical device of claim 1, wherein the skeleton structure is configured to have at least two bendable segments, each segment bendable by at least one driving wire.

8. A method of manufacturing a multi-layer outer cover for a bendable medical device wherein the bendable medical device comprises a plurality of guide rings at an interval forming a skeleton structure, comprising the steps of:
a. placing an inner polymer tube over the plurality of guide rings and the intervals between the guide rings in the skeleton structure;
b. compressing a length of a mesh tubular structure and placing the mesh tubular structure over the central lumen extrusion, wherein compression of the length of the mesh tubular structure increases the diameter of the mesh tubular structure in comparison to the neutral diameter of the mesh tubular structure;
c. releasing the compressing force and aligning the mesh tubular structure to fit over central lumen extrusion; and
d. placing an outer polymer tube over the mesh tubular structure to constrain the position and the length of the mesh tubular structure.

9. The method of claim 8, wherein each of the inner polymer tube, the mesh tubular structure, and the outer polymer tube (the layered tubes) are in an interference fit with the layer below.

10. The method of claim 9, wherein each layered tube can expand and contract along its length within the constraints of the next outer layered tube.

11. The method of claim 8, further comprising the step of:
cauterizing the ends of the mesh tubular structure prior to placing the outer polymer tube.

12. The method of claim 8, wherein the inner polymer tube is placed over the plurality of guide rings and the intervals between the guide rings in the skeleton structure and over a passive proximal tubular section which is proximal to the skeleton structure.

13. The method of claim 8, wherein the mesh is a braided polyester tube.

14. The method of claim 8, wherein the skeleton structure is configured to have at least two bendable segments, each segment bendable by at least one driving wire.

15. A medical apparatus made by the method of claim 8.

16. The bendable medical device of claim 1, wherein the inner polymer portion is a polymer tube and the outer polymer portion is a polymer tube which contacts the mesh tubular structure.

17. The bendable medical device of claim 1, wherein the inner polymer portion, the mesh tubular structure, and the outer polymer portion is an integral tube with the mesh tubular structure fully in-cased to form a smooth inner and a smooth outer surface.
